(19) United States
(12) Patent Application Publication
(10) Pub. No.: US 2014/0206966 A1
(43) Pub. Date: Jul. 24, 2014

(54) SENSOR

(71) Applicant: Google Inc., Mountain View, CA (US)

(72) Inventors: Zenghe Liu, Alameda, CA (US); Babak Amirparviz, Mountain View, CA (US)

(21) Appl. No.: 13/621,541

(22) Filed: Sep. 17, 2012

(51) Int. Cl. A61B 5/1477

(52) U.S. Cl. 600/345

(57) ABSTRACT

Apparatus, systems and methods employing contact lenses having an electrochemical sensor to detect ethanol concentration of a wearer of the contact lens are provided. In some aspects, a contact lens includes a substrate that forms at least part of a body of the contact lens and an electrochemical sensor, disposed on or within the substrate, that detects information related to concentration of alcohol present in blood of a wearer of the contact lens.

### TECHNICAL FIELD

This disclosure generally relates to a contact lens employing an electrochemical sensor on or within the contact lens to detect blood alcohol content of a wearer of the lens.

### BACKGROUND

Tests for determining blood alcohol content (BAC) of an individual are often necessary and useful in a variety of contexts. For example, BAC testing is important in connection with detecting and serving as a deterrent to drunk driving. Currently available BAC testing methods include direct blood sample testing and other indirect methods such as urine analysis and breath analysis. However, these methods suffer from a variety of drawbacks associated with difficulty in obtaining samples and high variability in test results.

### BRIEF DESCRIPTION OF THE DRAWINGS

FIG. 1A is an illustration of an exemplary non-limiting system that includes a contact lens employing an electrochemical sensor to detect information indicative of BAC of a wearer of the contact lens in accordance with aspects described herein.

FIGS. 1B and 1C depict enlarged perspectives of an example contact lens in accordance with aspects described herein.

FIG. 2 is an illustration of an example contact lens circuit for a contact lens employing an employing an electrochemical sensor to detect information indicative of BAC of a wearer of the contact lens in accordance with aspects described herein.

FIG. 3 is an illustration of an example contact lens having an electrochemical sensor to detect information indicative of BAC of a wearer of the contact lens in accordance with aspects described herein.

FIG. 4 is an illustration of an exemplary non-limiting reader device that receives from a contact lens, information indicative of BAC of a wearer of the contact lens in accordance with aspects described herein.

FIG. 5 is an exemplary flow diagram of a method that facilitates employing a contact lens to detect information indicative of BAC of a wearer of the contact lens in accordance with aspects described herein

FIG. 6 is an exemplary flow diagram of a method that facilitates receiving from a contact lens, information indicative of BAC of a wearer of the contact lens in accordance with aspects described herein.

FIG. 7 is an illustration of a schematic diagram of an exemplary networked or distributed computing environment with which one or more aspects described herein can be associated.

FIG. 8 is an illustration of a schematic diagram of an exemplary computing environment with which one or more aspects described herein can be associated.

### DETAILED DESCRIPTION

Various aspects are now described with reference to the drawings, wherein like reference numerals are used to refer to like elements throughout. In the following description, for purposes of explanation, numerous specific details are set forth in order to provide a more thorough understanding of one or more aspects. It is be evident, however, that such aspects can be practiced without these specific details. In other instances, structures and devices are shown in block diagram form in order to facilitate describing one or more aspects.

In one or more aspects, the disclosed subject matter relates to a contact lens. The contact lens can include a substrate that forms at least part of a body of the contact lens and an electrochemical sensor, disposed on or within the substrate, that detects information related to concentration of alcohol present in blood of a wearer of the contact lens. In an aspect, the electrochemical sensor is an amperometric sensor. The contact lens may further comprise a circuit disposed on or within the substrate that receives information related to concentration of alcohol present, and a transmitter that transmits the information to a reader.

In another aspect, a method is disclosed comprising using a contact lens comprising an electrochemical sensor that senses concentration of alcohol present in blood of a wearer of the contact lens. In an aspect, the electrochemical sensor comprises an amperometric sensor. The method can further comprise transmitting information related to the concentration of sensed alcohol to a reader.

In one or more additional aspects a device is presented comprising an interface component that interfaces with and receives from at least one contact lens, data relating to blood alcohol level of a wearer of the at least one contact lens. The device further includes an analyzing component that analyzes the received data and determines a blood alcohol level of the wearer of the at least one contact lens. The device can also employ a display component to generate a display corresponding to blood alcohol level of the wearer.

Apparatus, systems, and methods disclosed herein relate to contact lenses having an electrochemical sensor and circuitry to detect, determine, and report blood alcohol level of a wearer of the contact lens. In some aspects, the contact lens can transmit detected and/or determined data indicative of blood alcohol level of the wearer of the lens to a reader device. A reader device is thus further described that can receive transmitted information from the subject contact lens regarding blood alcohol level of the wearer of the contact lens and provide the information to a user.

The contact lens can comprise an electrochemical sensor integrated on or within a substrate of the contact lens. In an aspect, the electrochemical sensor can have two or three electrodes including but not limited to, a working electrode, a counter electrode, and a reference electrode respectively. In one aspect, the working electrode comprises a noble metal, such as platinum (Pt), that can directly oxidize alcohol (e.g. ethanol). In another aspect, the working electrode can contain a reduction-oxidation (redox) enzyme that specifically recognizes and oxidizes alcohol. With this aspect, the working electrode can also contain an electron transfer mediator that transfers electrons between the redox enzyme and the working electrode. In addition to the working electrode and counter electrode, the electrochemical sensor can include a reference electrode that controls the potential of the working electrode.

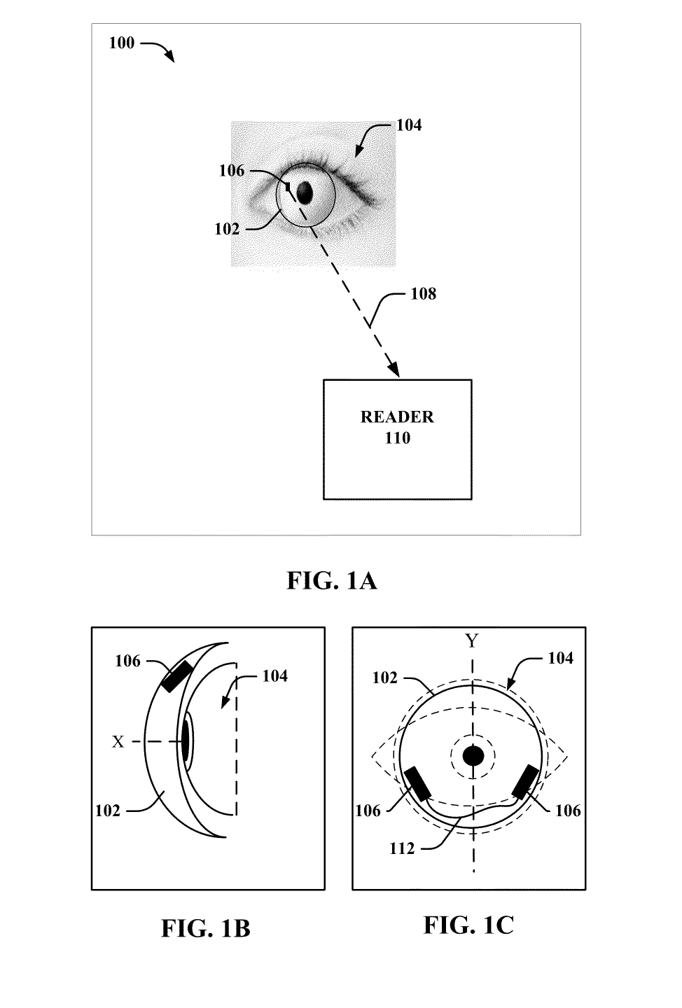

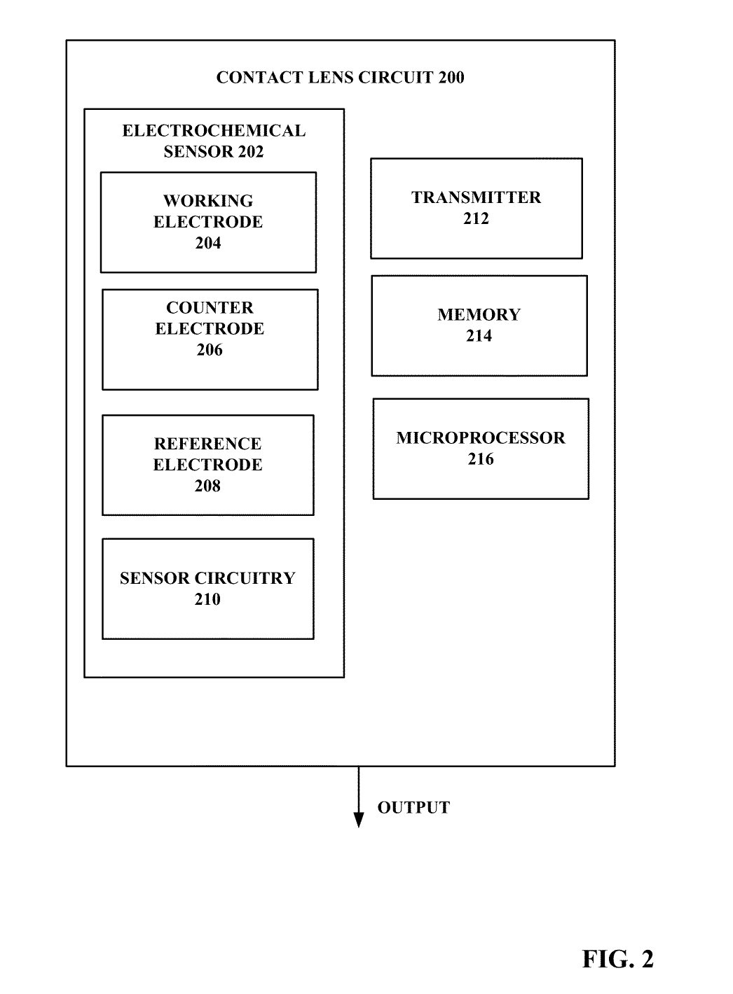

With reference now to the drawings, FIG. 1A is an illustration of an exemplary non-limiting system 100 that includes a contact lens 102 employing an electrochemical sensor that senses concentration of alcohol in blood of a wearer of the contact lens in accordance with aspects described herein. The system 100 includes a contact lens covering at least a portion of an eye 104 and having a contact lens circuit 106. The contact lens circuit 106 is described in greater detail with reference to FIG. 2. In an aspect, the contact lens circuit 106 includes an electrochemical sensor (not shown) that senses concentration of alcohol in blood of a wearer of the contact lens. In particular, the electrochemical sensor can detect concentration of ethanol generated by the wearer of the contact lens 102 by measuring potential (volts) and/or current (amperes) associated with a multi-electrode system in response to oxidation of the ethanol.

In various aspects, the contact lens 102 can detect one or more fluids on a surface of an eye and/or absorbed or otherwise received within the substrate of the contact lens. In particular, the contact lens 102 can detect ethanol in such fluids. In other aspects, the contact lens 102 can detect one or more gases, including ethanol gases, within the body of the contact lens or near a surface of the contact lens 102. The contact lens 102 can perform such detection via the electrochemical sensor. In particular, the electrochemical sensor can be located on and/or within a substrate of the contact lens. For example, the contact lens 102 can comprise a hydrogel substrate. One or more electrodes which make up part of the electrochemical sensor can further be located on and/or within the thickness of the hydrogel.

The electrochemical sensor can detect ethanol gas or fluid concentration present at the contact lens as a result of a current generated from oxidation of ethanol sensed at the sensor. In turn, the information gathered by the electrochemical sensor can be processed to determine blood alcohol content (BAC) of a wearer of the contact lens, either by a processor integrated within the contact lens or a processer associated with a reader device to which the information is transmitted.

For example, the electrochemical sensor can be an amperometric sensor that measures and uses the electrical current flowing between a working electrode and the counter electrode of the amperometric sensor to determine an analyte concentration of a sample. The value of the electrical current can depend on kinetics of a sensed chemical and electrochemical reactions as well as mass transfer rate of the analyte to the working electrode. When employed in connection with the subject contact lens 102, the amperometric sensor can determine concentration of ethanol fluid and/or gas on and/or within the contact lens substrate. For example, ethanol can be present in tear fluid reaching a surface of the contact lens and/or dispersing into the substrate of the contact lens (e.g. via a cavity on the lens and/or the hydrophilic nature of the substrate). In another example, ethanol vapors can be excreted by a wearer of the contact lens into the eye cavity. Such vapors can be sensed by the electrochemical sensor on and/or within the contact lens.

In an aspect, the electrochemical sensor can be integrated communicatively and physically with contact lens circuit 106. In other aspects, the contact lens circuit 106 can be separated physically and/or communicatively from the electrochemical sensor. For example, FIGS. 1B and 1C depict enlarged perspectives of an example contact lens 102 in accordance with aspects described herein. FIG. 1B depicts a cross-sectional view of contact lens 102 while FIG. 1C depicts a topical/planar view of contact lens 102.

As seen in FIG. 1B, contact lens circuit 106 is located within the substrate (e.g. within the thickness of the hydrogel) of the contact lens 102 and is depicted as a single unit. However, it should be appreciated that contact lens circuit 106 and/or one or more components associated with contact lens circuit 106 can be located on and/or within the substrate. According to this aspect, the contact lens circuit 106 and its associated components, including the electrochemical sensor, are co-located and communicatively coupled.

In another embodiment, as seen in FIG. 1C, one or more components of contact lens circuit 106 can be physically dispersed on and/or within the contact lens. For example, components of contact lens circuit 106 as presented in FIG. 1C are divided. According to this example, the electrochemical sensor can be physically separated from other components of the contact lens circuit. In other aspects, components of the electrochemical sensor, such as one or more electrodes, can be physically dispersed on and/or within the contact lens 102 substrate. In various embodiments, one or more components of the contact lens circuit 106, including the electrochemical sensor, can be communicatively coupled via one or more wires 112 and/or chemically.

Referring back to FIG. 1A, in some aspects, the contact lens 102 can include one or more components (not shown) to communicate sensed, detected and/or determined information, including but not limited to, the detected output current associated with oxidation of ethanol via the electrochemical sensor of the contact lens, the detected potential of the working electrode, or the determined blood alcohol concentration of the wearer of the contact lens. For example, the components can include a radio frequency (RF) antenna in some aspects. In some aspects, the information 108 can be communicated to a reader 110. In some aspects, the reader 110 can be an RF reader. Accordingly, the contact lens 102 can wirelessly communicate with a reader 110.

FIG. 2 is an illustration of a contact lens circuit 200 for a contact lens employing an electrochemical sensor 202 in accordance with aspects described herein. In various aspects, the contact lens circuit 200 can include one or more of the structure and/or functionality of the contact lens circuit 106 (and vice versa). Redundant description of like elements or components and associated functionality described herein in connection with respective embodiments of contact lenses, contact lens circuits, and electrochemical sensors is omitted for sake of brevity.

As shown in FIG. 2, the contact lens circuit 200 can include electrochemical sensor 202, transmitter 212, memory 214 and/or microprocessor 216. In some aspects and as depicted in FIG. 2, the contact lens circuit 200 includes electrochemical sensor 202 and associated components. In other aspects, electrochemical sensor 200 can be physically and/or communicatively independent (not shown) from the contact lens circuit 200. However, in various embodiments, one or more of electrochemical sensor 202, associated components 204-210, transmitter 212, memory 214 and/or microprocessor 216 can be electrically or chemically coupled to one another to perform one or more functions of the contact lens circuit 200.

In an aspect, electrochemical sensor 202 includes two or more electrodes including at least a working electrode 204 and a counter electrode 206. The electrochemical sensor can also employ additional electrodes, including a reference electrode 208. In some aspects, electrochemical sensor 202 can also include an electrolyte (not shown) and/or sensor circuitry 210. Functions of electrochemical sensor 202 can include sensing current between the working electrode and the counter electrode as result of oxidation of ethanol, detecting potential of the working electrode as a result of oxidation of ethanol, and outputting values associated with the sensed information. These values can then be employed by microprocessor 216, and/or an external processer associated with a reader device, in order to determine BAC of a wearer of a contact lens employing contact lens circuit 200.

The working electrode 204 is an electrode of electrochemical sensor 202 on which reaction of interest occurs. In particular, the working electrode facilitates oxidation of an analyte of interest (e.g., ethanol). The working electrode is used in conjunction with the counter electrode in a two electrode system, and further a reference electrode in a three electrode system. An oxidation reaction at the working electrode 204 results in flow of electrons from the working electrode 204 to counter electrode 206. In an aspect, sensor circuitry 210 facilitates this flow of electrons. In particular, electrons are generated from the oxidation of alcohol (ethanol) at the working electrode 204. These electrons then flow through circuitry 210 to the counter electrode 206, at which the electrons are transferred to an oxidant (e.g. oxygen) in the electrolyte solution separating the working and counter electrodes. The flow of electrons constitutes an electric current, which is proportional to analyte (e.g. ethanol) concentration. Reference electrode 208 can be employed to control the working electrode's potential and can serve as a reference for measuring potential of the working electrode. In an aspect, potential of the working electrode at a given point in time, or as averaged over a predetermined period of time, can indicate concentration of ethanol present in blood of a wearer of the contact lens.

In an aspect, the working electrode 204 comprises a noble or inert metal that can directly oxidize ethanol. For example, the working electrode can comprise but is not limited to: gold, silver or platinum. In another aspect, the working electrode can include an inert carbon such as glassy carbon or pyrolytic carbon. Still, in other aspects, the working electrode can employ a redox enzyme in addition to or in the alternative to a metal that can directly oxidize ethanol. In an aspect, the redox enzyme can specifically oxidize ethanol. For example, the redox enzyme can include a dehydrogenase, such alcohol dehydrogenase. As used herein, a dehydrogenase is an enzyme that oxidizes a substrate. However, it should be appreciated that any suitable enzyme or catalyst that can facilitate oxidation of alcohol can be employed by electrochemical sensor 202. In some aspects, where a redox enzyme or other type of alcohol oxidizing agent is employed, the electrochemical sensor 202 can also employ one or more electron transfer mediators to facilitate transfer of electrons received at the enzyme (as a result of oxidation of ethanol) to the working electrode. In an aspect, the counter electrode 206 can comprise any suitable material that can serve as an electron acceptor. For example, the counter electrode can comprise an electrochemically inert material such as gold, platinum, or carbon.

In an embodiment, the counter electrode 206 can be coupled between an electrolyte (e.g. a liquid electrolyte) and the working electrode 204. The working electrode 204 and counter electrode 206 can be in contact with the electrolyte in various aspects. According to this embodiment, the connection between the counter electrode 206 and the electrolyte enables electrical current to be applied to the working electrode 204 via the sensor circuitry. In particular, upon sensing ethanol, an electrochemical reaction can occur and the sensor circuitry 210 can generate an electrical current proportional to mass transfer rate of the ethanol.

In some aspects, contact lens circuit 200 further includes transmitter 212, memory 214 and/or microprocessor 216. Sensor circuitry 210 facilitates detection of a value (in amperes) of current between the working electrode 204 and the counter electrode 204 and/or a value of potential (in volts) of the working electrode as a result of oxidation of ethanol by the working electrode 204. The sensor circuitry 210 can further send sensed values to transmitter 212, memory 214, and/or microprocessor 216. In an aspect, transmitter 212 transmits sensed information (e.g. sensed current and/or working electrode potential values associated with sensed ethanol) to a reader device remote from the contact lens. For example, the transmitter 212 can include an RF antenna in some aspects. In turn, the reader device can perform analysis and processing of the detected current and/or working electrode potential values to determine blood alcohol level of a wearer of the contact lens.

In other aspects, microprocessor 216 can receive sensed information from the electrochemical sensor 202 and perform analysis and processing of the sensed information to determine a BAC of a wearer of a contact lens employing contact lens circuit 200. In turn, transmitter 212 can transmit the determined BAC to a reader.

Memory 214 can store computer-executable instructions for execution by microprocessor 216. Microprocessor 216 can execute computer-executable instructions to perform one or more functions of the contact lens circuit 200. Further, in some aspects, memory 214 can store any information sensed by electrochemical sensor 202 as well as predetermined information relating known current and/or working potential values to BAC levels. For example, memory 214 can store various look-up tables and/or algorithms relating potentially sensed information to BAC levels. Accordingly, microprocessor 216 can also employ various analytical techniques using sensed and stored information in order to determine a BAC level. For example, microprocessor 216 can convert a sensed output current received from electrochemical sensor 202 to a BAC value.

In an embodiment, microprocessor 216 (and/or an external processor) can employ various (explicitly or implicitly trained) classification schemes or systems (e.g., support vector machines, neural networks, expert systems, Bayesian belief networks, fuzzy logic, data fusion engines, etc.) in connection with performing analysis of sensed information. A classifier can map an input attribute vector, x=(x1, x2, x3, x4 . . . , xn), to a confidence that the input belongs to a class, such as by f(x)=confidence(class). Such classification can employ a probabilistic or statistical-based analysis (e.g., factoring into the analysis utilities and costs) to prognose or infer an action that a user desires to be automatically performed. A support vector machine (SVM) is an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used in this disclosure also is inclusive of statistical regression that is utilized to develop models of priority.

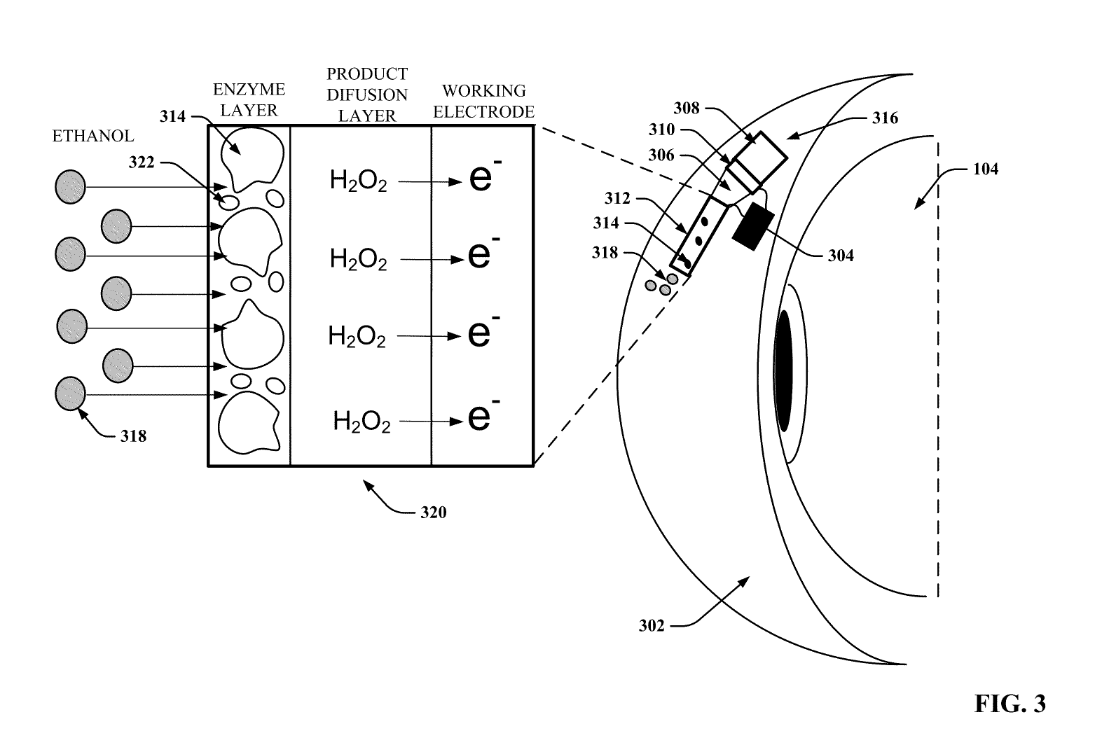

FIG. 3 is an illustration of an example contact lens 302 having an electrochemical sensor 316 for sensing BAC of a wearer of the contact lens in accordance with aspects described herein. In the exemplary contact lens 302, electrochemical sensor is 316 is physically separated from contact lens circuit 304 within the substrate body of the contact lens. Various components of the electrochemical sensor 316 and contact lens circuit 304 are electrically connected via one or more wires. In various aspects, the contact lens circuit 304 and electrochemical sensor 316 can include one or more of the structure and/or functionality of the contact lens circuits 106/200 and 202, respectively (and vice versa).

The electrochemical sensor 316 includes a working electrode 312, a counter electrode 310, and a reference electrode 308. The working electrode 312 is separated from the counter electrode 310 by an electrolyte or electrolyte solution 306 and the working electrode 312 is connected to the counter electrode 310 via circuitry held in contact lens circuit 304. In an aspect the working electrode comprises a noble metal, such as platinum. For example, in an aspect, the working electrode 312 can form a compartment or channel where an inner lining of a housing of the compartment or channel is doped with or coated with platinum. In some aspects, the working electrode can include one or more enzymes 314 (e.g. alcohol oxidase or alcohol deyhdrogenase) and one or more mediators 322 that improve electron transfer kinetics. Ethanol 318 can further enter the channel to come into contact with reactive elements of the working electrode. Accordingly, when ethanol 318 enters the working electrode channel 312, it will be oxidized.

In an aspect, the working electrode channel 312 can include one or more selective diffusion barriers that selectively allow substances to pass. For example, the working electrode channel 312 can include a barrier layer material that selectively allows ethanol to enter the channel 312 while inhibiting other substances from entering the channel. The working electrode channel 312 can also include various barrier layers that serve as a proton exchange membrane. In particular, as ethanol entering the working electrode channel 312 becomes oxidized, protons and/or water, or other products such as hydrogen peroxide produced can be transported across a product diffusion layer.

An exemplary view of functional aspects of the working electrode channel 312 is presented in diagram 320. As illustrated in diagram 320, ethanol enters the working electrode channel to come into contact with an enzyme layer containing an enzyme such as alcohol oxidase that catalyses the oxidation of ethanol to hydrogen peroxide. The hydrogen peroxide produced in the working electrode as a result of ethanol oxidation is passed through a product diffusion layer, and electrons are generated at the working electrode.

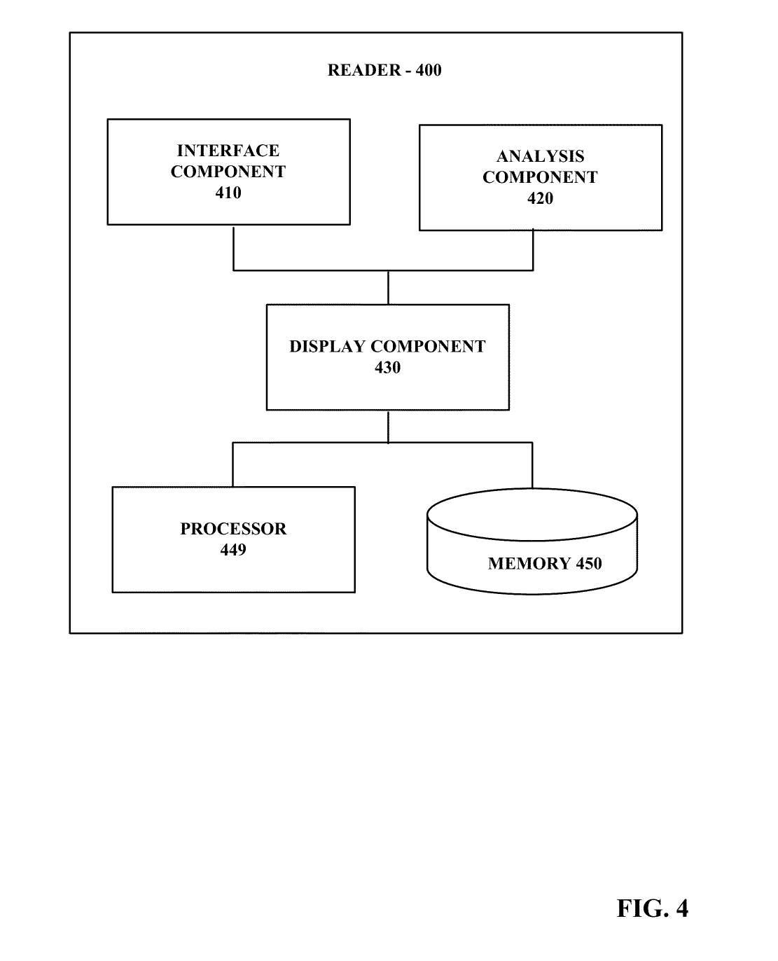

FIG. 4 is an illustration of an exemplary non-limiting reader device 400 that interfaces with a contact lens employing an electrochemical sensor to receive information indicative of concentration of alcohol in the blood of a wearer of the contact lens in accordance with aspects described herein. In various aspects, the reader device 400 can include one or more of the structure and/or functionality of reader device 110 (and vice versa).

As shown in FIG. 4, reader device 400 can include interface component 410, analysis component 420, and display component 430. Aspects of device 400 constitute machine-executable components embodied within machine(s), e.g., embodied in one or more computer readable mediums (or media) associated with one or more machines. Such components, when executed by the one or more machines, e.g., computer(s), computing device(s), virtual machine(s), etc. can cause the machine(s) to perform the operations described. Device 400 can include memory 450 for storing computer executable components and instructions. A processor 440 can facilitate operation of the computer executable components and instructions by device 400.

Interface component 410 interfaces with and receives from at least one contact lens, data relating to blood alcohol level of a wearer of the contact lens. In particular, interface component 410 can interface with contact lenses described herein that comprise a contact lens circuit such as contact lens circuit 106, 200 and/or 304. In an aspect, interface component 310 employs a receiver, such as an RF receiver, to receive sensed and/or determined information from a contact lens comprising a contact lens circuit as described herein. In some aspects, interfacing component 410 can receive a determined value indicating a BAC of a wearer of the contact lens from which the information was transmitted. According to this aspect, the contact lens can include appropriate circuitry and components to process data sensed by an electrochemical sensor thereon and/or therein.

In another aspect, the reader can receive raw data from a contact lens relating to a sensed concentration of ethanol. For example, the interface component 410 can receive one or more values indicating an intensity of a current (e.g. in amperes) resulting from the oxidation of ethanol by an electrochemical sensor on and/or within the contact lens. In another example, the interface component 410 can receive one or more values indicating a potential (e.g. in volts) of a working electrode employed by an electrochemical sensor on and/or within the contact lens. According to this embodiment, the reader 300 comprises an analysis component 420 that can analyze the received raw data and determine BAC of a wearer of the contact lens. For example, the analysis component 420 can employ information in memory 440 that relates the received information to BAC level content. The reader device can further include a display component 430 that generates a display corresponding to blood alcohol level of the wearer.

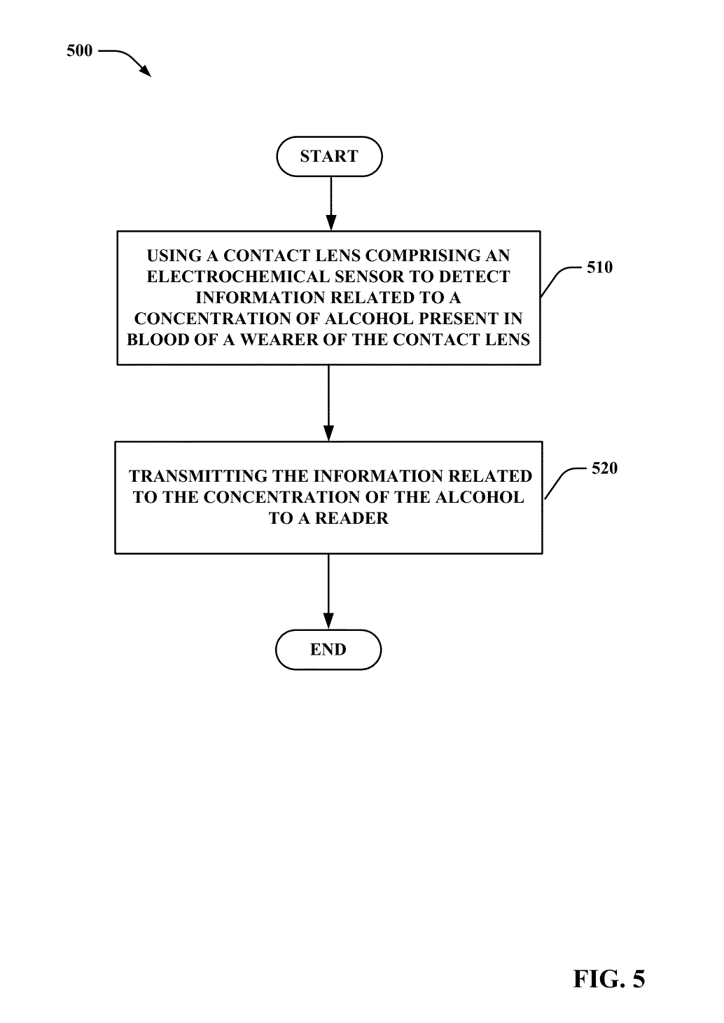

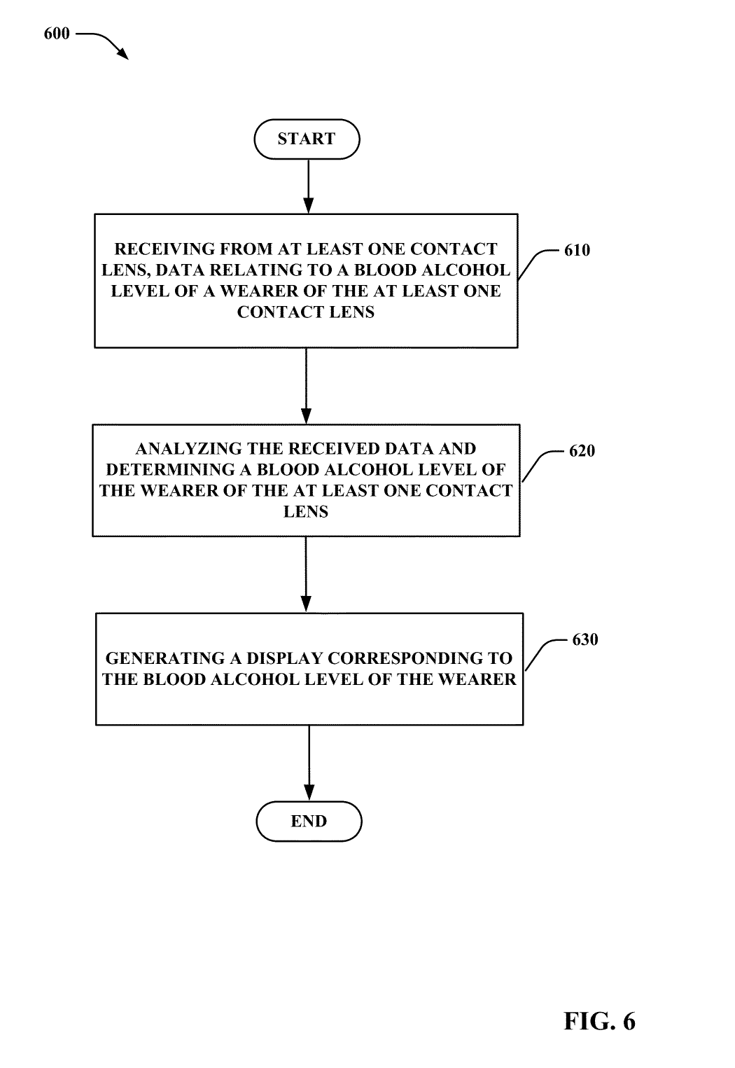

FIGS. 5-6 illustrates methodologies or flow diagrams in accordance with certain aspects of this disclosure. While, for purposes of simplicity of explanation, the methodologies are shown and described as a series of acts, the disclosed subject matter is not limited by the order of acts, as some acts can occur in different orders and/or concurrently with other acts from that shown and described herein. For example, those skilled in the art will understand and appreciate that a methodology can alternatively be represented as a series of interrelated states or events, such as in a state diagram. Moreover, not all illustrated acts may be required to implement a methodology in accordance with the disclosed subject matter. Additionally, it is to be appreciated that the methodologies disclosed in this disclosure are capable of being stored on an article of manufacture to facilitate transporting and transferring such methodologies to computers or other computing devices.

Referring now to FIG. 5, presented is a flow diagram of an example application of systems and apparatuses disclosed in this description in accordance with an embodiment. In an aspect, in exemplary methodology 500, a contact lens such as those described herein (e.g. 102 and the like) is employed to detect information pertaining to blood alcohol level of the wearer of the contact lens. At 510, a contact lens comprising an electrochemical sensor (e.g. contact lens 102, 302) is used to detect information related to a concentration of alcohol present in blood of a wearer of the contact lens, (e.g. using contact lens circuit 106, 200 or 304). At 520, the detected information related to the concentration of the alcohol is transmitted to a reader (e.g. using transmitter 212).

Turning now to FIG. 6, a method 600 can include receiving information detected by a contact lens relating to blood alcohol level of the wearer of the contact lens (e.g. using reader device 110 or 400). At 610 data related to a blood alcohol level of a wearer of a contact lens is received from the contact lens (e.g. using interface component 410). At 620, the received data is analyzed and the blood alcohol level of the wearer of the contact lens is determined (e.g. using analysis component 420). At 630, a display is generated corresponding to the blood alcohol level of the wearer (e.g. using display component 430).

### Exemplary Networked and Distributed Environments

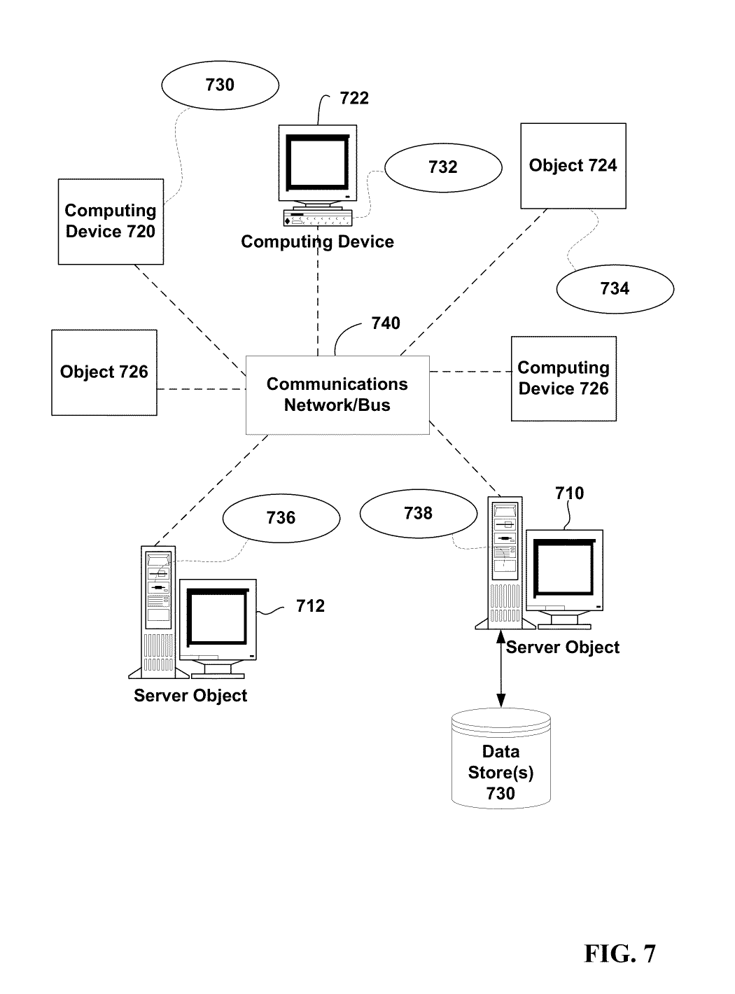

FIG. 7 provides a schematic diagram of an exemplary networked or distributed computing environment with which one or more aspects described in this disclosure can be associated. The distributed computing environment includes computing objects 710, 712, etc. and computing objects or devices 720, 722, 724, 726, 728, etc., which can include programs, methods, data stores, programmable logic, etc., as represented by applications 730, 732, 734, 736, 738. It can be appreciated that computing objects 710, 712, etc. and computing objects or devices 720, 722, 724, 726, 728, etc. can include different devices, such as active contact lenses (and components thereof), personal digital assistants (PDAs), audio/video devices, mobile phones, MPEG-1 Audio Layer 3 (MP3) players, personal computers, laptops, tablets, etc.

Each computing object 710, 712, etc. and computing objects or devices 720, 722, 724, 726, 728, etc. can communicate with one or more other computing objects 710, 712, etc. and computing objects or devices 720, 722, 724, 726, 728, etc. by way of the communications network 740, either directly or indirectly. Even though illustrated as a single element in FIG. 7, network 740 can include other computing objects and computing devices that provide services to the system of FIG. 7, and/or can represent multiple interconnected networks, which are not shown.

In a network environment in which the communications network/bus 740 can be the Internet, the computing objects 710, 712, etc. can be Web servers, file servers, media servers, etc. with which the client computing objects or devices 720, 722, 724, 726, 728, etc. communicate via any of a number of known protocols, such as the hypertext transfer protocol (HTTP).

### Exemplary Computing Device

As mentioned, advantageously, the techniques described in this disclosure can be associated with any suitable device. It is to be understood, therefore, that handheld, portable and other computing devices (including active contact lens having circuitry or components that compute and/or perform various functions). As described, in some aspects, the device can be the contact lens (or components of the contact lens) and/or the reader described herein. In various aspects, the data store can include or be included within, any of the memory described herein, any of the contact lenses described herein and/or the reader device described herein. In various aspects, the data store can be any repository for storing information transmitted to or received from the contact lens.

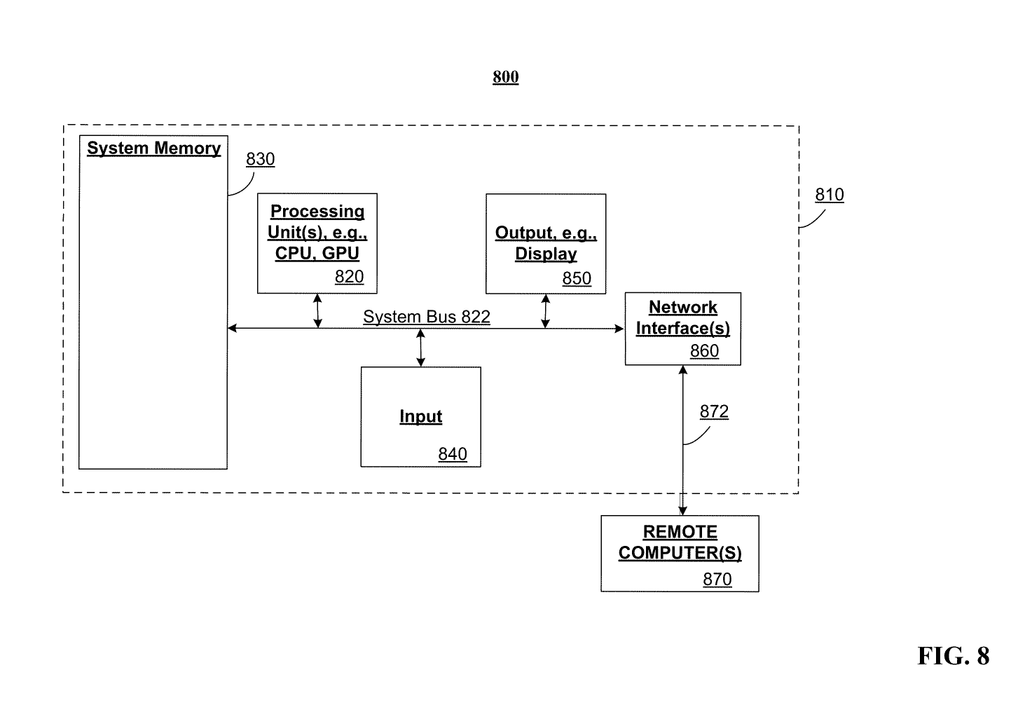

FIG. 8 illustrates an example of a suitable computing system environment 800 in which one or aspects of the aspects described in this disclosure can be implemented. Components of computer 810 can include, but are not limited to, a processing unit 820, a system memory 830, and a system bus 822 that couples various system components including the system memory to the processing unit 820.

Computer 810 typically includes a variety of computer readable media and can be any available media that can be accessed by computer 810. The system memory 830 can include computer storage media in the form of volatile and/or nonvolatile memory such as read only memory (ROM) and/or random access memory (RAM). By way of example, and not limitation, memory 830 can also include an operating system, application programs, other program components, and program data.

A user can enter commands and information into the computer 810 through input devices 840 (e.g., keyboard, keypad, a pointing device, a mouse, stylus, touchpad, touch screen, motion detector, camera, microphone or any other device that allows the user to interact with the computer 810). A monitor or other type of display device can be also connected to the system bus 822 via an interface, such as output interface 850. In addition to a monitor, computers can also include other peripheral output devices such as speakers and a printer, which can be connected through output interface 850.

The computer 810 can operate in a networked or distributed environment using logical connections to one or more other remote computers, such as remote computer 860. The remote computer 860 can be a personal computer, a server, a router, a network PC, a peer device or other common network node, or any other remote media consumption or transmission device, and can include any or all of the elements described above relative to the computer 810. The logical connections depicted in FIG. 8 include a network 870, such local area network (LAN) or a wide area network (WAN), but can also include other networks/buses e.g., cellular networks.

Computing devices typically include a variety of media, which can include computer-readable storage media and/or communications media, in which these two terms are used herein differently from one another as follows. Computer-readable storage media can be any available storage media that can be accessed by the computer, can be typically of a non-transitory nature, and can include both volatile and nonvolatile media, removable and non-removable media. By way of example, and not limitation, computer-readable storage media can be implemented in connection with any method or technology for storage of information such as computer-readable instructions, program components, structured data, or unstructured data. Computer-readable storage media can include, but are not limited to, RAM, ROM, electrically erasable programmable read only memory (EEPROM), flash memory or other memory technology, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium. In various aspects, the computer-readable storage media can be, or be included within, the memory, contact lens (or components thereof) or reader described herein.

On the other hand, communications media typically embody computer-readable instructions, data structures, program components or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals.

It is to be understood that the aspects described in this disclosure can be implemented in hardware, software, firmware, middleware, microcode, or any combination thereof. For a hardware aspect, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors and/or other electronic units designed to perform the functions described in this disclosure, or a combination thereof.

For a software aspect, the techniques described in this disclosure can be implemented with components or components (e.g., procedures, functions, and so on) that perform the functions described in this disclosure. The software codes can be stored in memory units and executed by processors.

What has been described above includes examples of one or more aspects. It is, of course, not possible to describe every conceivable combination of components or methodologies for purposes of describing the aforementioned aspects, but one of ordinary skill in the art can recognize that many further combinations and permutations of various aspects are possible. Accordingly, the described aspects are intended to embrace all such alterations, modifications and variations that fall within the spirit and scope of the appended claims.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

The aforementioned systems have been described with respect to interaction between several components. It can be appreciated that such systems and components can include those components or specified sub-components. Sub-components can also be implemented as components communicatively coupled to other components rather than included within parent components (hierarchical). Additionally, it is to be noted that one or more components can be combined into a single component providing aggregate functionality. Any components described in this disclosure can also interact with one or more other components not specifically described in this disclosure but generally known by those of skill in the art.

In view of the exemplary systems described above methodologies that can be implemented in accordance with the described subject matter will be better appreciated with reference to the flowcharts of the various figures. While for purposes of simplicity of explanation, the methodologies are shown and described as a series of blocks, it is to be understood and appreciated that the claimed subject matter is not limited by the order of the blocks, as some blocks can occur in different orders and/or concurrently with other blocks from what is depicted and described in this disclosure. Where non-sequential, or branched, flow is illustrated via flowchart, it can be appreciated that various other branches, flow paths, and orders of the blocks, can be implemented which achieve the same or a similar result. Moreover, not all illustrated blocks may be required to implement the methodologies described in this disclosure after.

In addition to the various aspects described in this disclosure, it is to be understood that other similar aspects can be used or modifications and additions can be made to the described aspect(s) for performing the same or equivalent function of the corresponding aspect(s) without deviating there from. Still further, multiple processing chips or multiple devices can share the performance of one or more functions described in this disclosure, and similarly, storage can be provided across a plurality of devices. The invention is not to be limited to any single aspect, but rather can be construed in breadth, spirit and scope in accordance with the appended claims.

What is claimed is:

1. A contact lens, comprising:
a substrate that forms at least part of a body of the contact lens;
an electrochemical sensor, disposed on or within the substrate, that is configured to sense information related to concentration of alcohol present in blood of a wearer of the contact lens when the contact lens is worn.

2. The contact lens of claim 1, wherein the electrochemical sensor comprises an amperometric sensor.

3. The contact lens of claim 1, wherein the electrochemical sensor comprises at least two electrodes including a working electrode and a counter electrode.

4. The contact lens of claim 3, wherein the electrochemical sensor is configured to sense the information by sensing an electrical current generated between the at least two electrodes in response to oxidation of ethanol.

5. The contact lens of claim 3, wherein the working electrodes comprises a noble metal.

6. The contact lens of claim 3, wherein working electrode comprises platinum (Pt).

7. The contact lens of claim 3, wherein the working electrode is configured to directly oxidize ethanol.

8. The contact lens of claim 3, wherein the working electrode comprises an enzyme that oxidizes ethanol.

9. The contact lens of claim 8, wherein the enzyme includes at least one of alcohol dehydrogenase or alcohol oxidase.

10. The contact lens of claim 8, wherein the electrochemical sensor further comprises an electron transfer mediator that is configured to shuttle electrons from the enzyme to the working electrode.

11. The contact lens of claim 3, wherein an additional reference electrode is configured to control the working electrode's potential and serve as a reference for measuring the potential of the working electrode.

12. The contact lens of claim 1, further comprising:
a circuit disposed on or within the substrate that is configured to receive the information; and
a transmitter that is configured to transmit the information to a reader.

13. The contact lens of claim 12, further comprising a processor that is configured to determine or infer blood alcohol level of the wearer based on the information.

14. A method comprising:
using a contact lens comprising an electrochemical sensor to determine or infer concentration of alcohol present in blood of a wearer of the contact lens.

15. The method of claim 14, further comprising using an amperometric sensor to measure electrical current and determine analyte concentration of a sample.

16. The method of claim 15, wherein the determination or inference is based at least in part on electrical current generated between at least two electrodes of the electrochemical sensor in response to oxidation of ethanol.

17. The method of claim 16, comprising using an enzyme that catalyzes ethanol oxidation.

18. The method of claim 17, wherein the enzyme includes at least one of alcohol dehydrogenase or alcohol oxidase.

19. The method of claim 14, further comprising transmitting information related to the determined or inferred blood alcohol concentration to a reader.

20. A device, comprising:
a memory that stores computer executable components; and
a processor that executes the following computer executable components stored in the memory:
an interface component configured to interface with and receive from at least one contact lens, data relating to blood alcohol level of a wearer of the at least one contact lens;
an analyzing component configured to analyze the received data and determine blood alcohol level of the wearer of the at least one contact lens; and
a display component configured to generate a display corresponding to the blood alcohol level of the wearer.
